(19) United States
(12) Patent Application Publication
(10) Pub. No.: US 2023/0009416 A1
(43) Pub. Date: Jan. 12, 2023

(54) NOVEL NANOTECHNOLOGY-DRIVEN PROTOTYPES FOR AI-ENRICHED BIOCOMPATIBLE PROSTHETICS FOLLOWING EITHER RISK OF ORGAN FAILURE OR MODERATE TO SEVERE IMPAIRMENT

(71) Applicant: GLAUCIA PEREIRA, BRISTOL (GB)

(72) Inventor: GLAUCIA PEREIRA, BRISTOL (GB)

(21) Appl. No.: 17/756,828

(22) PCT Filed: Dec. 11, 2020
(86) PCT No.: PCT/GB2020/000100

§ 371 Date: Jun. 3, 2022

(30) Foreign Application Priority Data
Dec. 11, 2019 (AT) A 60273/2019

(51) Int. Cl. A61M 60/191; A61M 60/515; A61M 60/187; A61M 60/289; A61M 60/268; B33Y 80/00

(52) U.S. Cl. CPC A61M 60/191; A61M 60/515; A61M 60/187; A61M 60/289; A61M 60/268; B33Y 80/00; A61M 2205/054; A61M 2207/00; A61M 2205/0244

(57) ABSTRACT

Three groups of biocompatible implants were created, to leverage physiological impairment caused by (i) cardiovascular, (ii) renal, and (iii) neuronal diseases. Each group of implants is subdivided into three categories according to extra functionality added plus integrated additions. The first generation contains basic functionality and the second and third generations contain extra functions. Finally, further additions can be combined and integrated. Therefore, the first group comprises of the “First Generation of Cardiovascular Implants” plus the “Second Generation of Cardiovascular Implants” plus the “Third Generation of Cardiovascular Implants” plus additional integrations named “Additions”. Equally, the second group comprises of the “First”, the “Second” and the “Third” Generation of Renal Prosthetics plus Additions. The same categorisation applies to Neural Implants, which are three generations plus additions. This can be found in the description of claims presented in the Austrian Prio (provisional patent application) number A 60273/2019, from 11 Dec. 2019.

### BACKGROUND OF THE INVENTION

Genetic Engineering and Tissue Bioengineering will certainly progress towards overcoming current challenges in Organ Donation and Transplantation. Engineered organs deriving from stem cell-based engineering of recipients' tissue is potentially the future of organ donation and transplantation. However, although the technology is already known, achieving the desirable reproducible well-controlled results may be long-term endeavour, with genetic engineering (e.g., CRISPR-Cas9) adding value to the former.

For the time being, the here presented invention proposes feasible alternatives to genetic engineering, to leverage the significant burden of mortality caused by the imbalance in the number of donor-recipient and the needed physiological compatibility. Observing the success of work in synthetic biology, whose findings can be found in the current literature, it is a widely held view that biocompatible materials and artificially driven bioprocesses are of great value to biomedicine. Therefore, the inventor here proposes combining biocompatible materials to shape desirable implants/prosthetics (soft matrix based on silicone elastomers and polymers due to their flexibility, adjustability, high compressibility, biocompatibility, and resistance to mechanical forces) and biocompatible flexible electronic microchips/sensors. This, combined with existing technology for medical devices, to create a dynamic engine (a biocompatible soft-hardware coupling) that emulates human organs. The description is presented in the Austrian Prio A60273/2019.

Via specialised software driven by AI-technology and fed by multiple sensors. This dynamic biocompatible engine counts on functional and security mechanisms, which remotely communicate with external platforms (computers located in hospitals and mobile phones) that allow clinical specialists and technical professionals to maintain the biocompatible modules, reacting effectively in case of fault. The description is presented in the Austrian Prio A60273/2019.

Primarily, the composite technology here proposed does not intend to replace bio-tissue long-term basis but rather sustain life, safely, assuring that the recipient will reach the beginning of the queue for a compatible donor. Ultimately, whether the circumstances permit, long-term implants would be employed—e.g. the second generation of renal prosthetics presented in presented in the Austrian Prio A60273/2019, which would replace an entire organ, supporting the second biological kidney, following lateral nephrectomy, reducing the workload that the existing kidney is subject to. Evidently, existing technology in material science, computing, sensors, electronics, and nanocomposites is well tested and designed to successfully support the developments here proposed, safely. Therefore, both the current literature and the inventor's results for the tested prototypes suggest that this invention has an immense potential for large-scale manufacturing and future use in clinics.

These prototypes were envisioned by the author, back in 2006, while applying AI-technology to both model and forecast pathological ECG and EEG patterns associated to epileptic episodes and cardiovascular fibrillation, respectively. The initial design was refined over more than a decade of research in biomedical physics and computational systems engineering. Therefore, this invention uses physiological signals captured via sensors, transmits them to be analysed by AI-based software, and feedbacks microchips working as actuators that control de prosthetics. Among the additions to the basic sets of functionalities, resulting in the third generation of implants, are delivery systems based on nanotechnology that can control therapeutics—e.g. delivery of medicinal compounds and injections of regenerative stem cells composites (Austrian Prio A60273/2019).

Additional functionalities related to safety and quality assurance are added, as a set of sensors that send signals to a “monitor/display” in the host patient (e.g. located in the wrist). The simplest version would contain three indicative lights: (i) green indicating that the implant is fully functional and that local homeostasis is preserved, (ii) yellow indicating that the sensors are indicating an imminent need for inspection, (iii) red indicating severe risk of failure. This advanced notice would allow time for action to be taken, safeguarding the wellbeing of the patient (Austrian Prio A60273/2019). Therefore, this invention rely on multiple disciplines: genetic engineering and material science knowledge, improving the perspectives given by mathematics and computing.

Sensors and Material Science: In the Tech-Bio interplay, proper automation in a dynamically controlled environment needs sensors. These elements detect, capture, and convert non-electrical stimulus into electrical signals that are conveniently transmitted to computational platforms, to be processed and analysed. Indeed, in bioengineering, in-silico analyses of physical and chemical stimulus unveiling central material properties and signalling cascades are crucial for modelling and forecasting trends that are either not easily replicated in a well-controlled experimental environment or require further data processing. The current literature in the field of biotechnology is vast (Moglia et al. 2009; Canales et al. 2015; Pereira 2017a, 2019; Pereira et al. 2019; Li et al. 2019). Regarding sensors, in (Boutry et al. 2019), the authors proposed a biodegradable alternative to conventional wired implantable monitoring systems, following regenerative cardiovascular medical interventions that imply the imminent need to dynamically monitor patients' signals. The proposed biodegradable wireless flexible arterial-pulse sensor was designed to be degraded by the host physiological system, eliminating post-surgery interventions for monitoring systems' removal. Similarly, Shin et al. (Shin et al. 2019) designed a biodegradable pressure sensor for diagnosis and follow-up of disease triggered by intracranial, intraocular or intravascular abnormal pressure (e.g., traumatic brain injury, glaucoma and hypertension). Progress in material science has enabled longer device lifespan, reducing both biocompatibility drawbacks and device surgical removal risks; increasing the probability of success in clinical applications. Indeed, material properties advancing bio-devices manufacturing have been the subject of a considerable amount of research (Seignez and Phillipson 2017; Bank 2019; Bannerman et al. 2019), particularly in neuroscience; with Liu et al (Liu et al. 2019b) discussing ion and electronic conductivity, along with an idealised coefficient of elasticity that optimises probe design for artificial neuronal stimulation, reducing inconveniences posed by the biocompatibility of microelectronics. Microelectronics is proven challenging when biocompatibility is a central requirement. At nanoscale, the inherent drawbacks could increase further and potential solutions may indicate diverse directions.

As indicated in paragraphs 0002, 0003 and 0006, this invention is based on long lifespan biocompatible materials—Austrian Prio A60273/2019; (silicone elastomers and polymers for membranes' construction, along with super-hydrophobic silicone/polytetrafluoroethylene films for encapsulation of implantable microelectronics), in contrast with the biodegradable materials described in paragraph 0007, which are commonly found in the current literature.

Nanotechnology Empowering Implanted Prosthetics: Many scholars hold the view that nanomaterials may revolutionise the future of biomedicine. In view of latest progress in the field, it seems reasonable to attribute to nanotechnology such high expectations. Nanoparticles are promising drug delivery systems (Conte et al. 2017; Singh et al. 2019; Pereira 2020c), leveraging drug solubility shortcomings, optimising compounds distribution, and improving therapeutic results by better targeting specific tissue; while benefitting from minimally invasive nanoparticles' distribution techniques (e.g., magnetic fields driving superparamagnetic metal nanoparticles and core-shell nanoparticles within the human body). The prospects for nano-sensors are equally remarkable (Liu et al. 2019a; Masvidal-Codina et al. 2019; Tite et al. 2019; Pereira 2020c). Indeed, the development of graphene nano-complexes has empowered bioelectronics, conceiving adaptive implant-tissue interfaces, reducing the risk of immune toxicity-driven side effects (e.g., battery-less photodynamically activated bioelectronics (Liu et al. 2015)).

Within these perspectives, this invention is based on both encapsulated microelectronics and biosensors designed using graphene transistors and electrodes (e.g., using high conductive graphene-metal nano-composites), taking into account existing technological limitations. In (Pereira 2020c), the inventor discussed major trends in nanotechnology-related drug development and the success of phytotherapeutics. The rather complex compromise drug-effectiveness and reducing invasiveness was assessed and both nano techniques and materials (e.g., liposomes, dendrimers, and nano-emulsions) were presented under the perspective of drug solubility, stability and bioavailability. In that work, biomimetics were discussed in the context of the replication of living systems protective mechanisms (e.g., natural self-healing modelled as self-medication via drug delivery systems); which would ultimately set directions in synthetic tissue engineering.

Here, the inventor presents implanted prosthetics' designs that emulate the target tissue structure and functionality, as indicated in paragraph 0010, because such features greatly justify biocompatible synthetic tissue engineering in the context of organ donation and transplantation applications. Additionally, in (Pereira 2020c), the inventor discussed a proposed biocompatible quantum implant, whose design is intended to leverage neuronal impairment, resulting from chronic diseases like the Alzheimer's disease. Similarly, here, the inventor focus on biocompatible structures that couple with the human tissue, in order to leverage functional impairment—cardiovascular, renal, and neural implants (Austrian Prio A60273/2019).

Artificial Intelligence Orchestrating Signal-Response-driven Bioactivity: Artificial Intelligence (AI) is a field of knowledge that turned out to become increasingly popular over the last two or three decades. AI's driven mechanisms flourished in early 90's, experiencing rapid growth in the last decade. In biomedicine, AI is proving to be of much assistance, following a vast amount of the current literature reporting on successful neural signals recording and translation into speech, using AI-based data processing (Anumanchipalli et al. 2019; Akbari et al. 2019). The benefits are diverse, from assistance to patients suffering from severe muscular atrophy (e.g., spinal muscular atrophy) and defects in the laryngeal nerve (e.g., vocal cord paralysis) to therapeutic follow-up and assessment of disease progression in non-resident nervous system impairment (e.g., dysarthria caused by either brain tumour or stroke). From LSTM's to ConvNets, Deep Learning conveys algorithms capable of breaking down large signals (e.g., ECG time series and 3D image signals), preserving structural coherence, forecasting trends (e.g., predicting pathological spikes in ECG) and reconstructing morphological data (e.g. filtering noisy 3D images). New features granted by AI technology are of great value for monitoring vital signals (Gholami et al. 2018), for tracking biomarkers via the assessment of biochemical signalling cascades (Pereira 2017a, 2020a; Pereira et al. 2019), and for disease prognosis, diagnosis, and follow-up (Kumar et al. 2020; Nagarajan and Sathish Kumar 2020). Undoubtedly, AI techniques are equally important in facilitating the development of new therapies (Pereira 2017b, 2019, 2020b; Pereira et al. 2019).

Knowledge generation via adversarial nets and rules inference using reinforcement learning are at the forefront of high-tech (Pereira 2017c, 2019, 2020a). In biomedicine, these techniques can greatly enrich hybrid systems, as the ones here proposed. To exemplify, LSTM's can empower systems with real time forecasting capabilities, using streaming data transmitted by sensors; and reinforcement learning can decide how to play the biological game, in sceneries where prior knowledge for rules generation is unknown.

The above indicated techniques (paragraphs 0012 and 0013) are applied to enrich this invention. LSTM forecasts ECG progress, indicating the likelihood of a given spike, in real time. At this stage, there is no complete historical data for supervised learning algorithms, to determine the best course of action. In such case, reinforcement learning dynamically learns the know-how in responding to forecasted stimulus (LSTM-generated ECG signals) and adversarial nets additionally improves the prescribed actions, by simulating a signal-response concurrent automaton (in analogy to the concept of Turing machines), which here translates into knowledge generation used to excel the system response to physiological stimulus. For example, this combined AI structure combines both a given LSTM signal and the inherent risk of implanted prosthetics failure that was estimated by reinforcement learning mechanisms, ultimately determining the best course of action, to mitigate risk.

Current Challenges in Organ Donation and Transplantation: End state organ failure inexorably results in imminent need of organ transplantation—e.g. kidney transplantation is a generally accepted remedy for assuring both life quality and cost effectiveness, in comparison with long-lasting dialysis. Organ donation and transplantation are decisive in the global health system. According to the NHSBT annual statistics (NHSBT 2017), the number of successful donations in the UK from 2016 to 2017 stood for about 2,777. Among these figures, 1,163 donations resulted from donors enduring circulatory death. Additionally, c. 58% of donations resulted mainly from brain arrest, representing about 72% increase in 15 years, in comparison to the numbers for cadaveric solid organ donors published in 2002-2003 (NHSBT 2003). Furthermore, in 2015/16, the British NHS system experienced a peak in deceased donors and the number of successful transplantations was the highest in history. The above figures resulted from collective efforts from professionals working in the field, along with an increasing number of donor consents. Worldwide, efforts towards improving organ donation and transplantation mechanisms are equally important, being indirectly measured via survival rate, following organ transplantation. One single example is found in information published by the World Health Organization (WHO), which shows one year survival rates, following kidney transplantation, worldwide (WHO 2019). However, the perspectives are still far from ideal, considering the number of critically ill patients in waiting lists.

In December 2019, the NHS UK reported on 6,176 individuals waiting for a donor (NHSBT 2019a), with the number of patients in need of a heart transplant increasing by c. 134%, from 2010 to 2018/19— NHSBT Transplant Activity Report 2018/19, page 62 (NHSBT 2019b). In conjunction with that, a remarkably high number of patients suffering from organ failure tend not to reach the end of the process, vanishing before organ transplantation (Statista 2019). Indeed, in 2019, in the UK alone, a significant number of individuals died waiting for a kidney transplant, which is not thought to be the most challenging donor-recipient match case. In 2014, US authorities revealed that 17,107 kidney transplants took place in the country, with a predominant number of organs retrieved from deceased donors (11,570). In late 2016, the same authorities reported on 121,678 individuals waiting for a kidney transplant (NKF 2016). Now, in 2019, in the US, c. 84% of the recipients are waiting for a kidney (Health Resources & Services Administration 2019). Ultimately, in 2016, authorities in South Africa reported on only 512 organ and cornea transplantations (Organ Donor Foundation 2016), which may indicate that many are still in need; assuming a population of about 57 million (informed in 2017 by the World Bank ONS UK).

In Southern Asia, the figures are equally critical (Chan-on and Sarwal 2017), with the number of end state organ failure cases increasing at inverse proportion with the decreasing number of successful transplantations. The Southern Asian figures show trends far more critical than what is observed worldwide. Indeed, according to (Chan-on and Sarwal 2017), in 2014, the statistic indicate that the number of organ transplantation per million population in the region was c. 88% lower than that in the US and c. 86.4% lower than in Europe. This, among a population of about 1.9 billion inhabitants, against c. 327 million in the US and c. 741 million in Europe.

In this scenery, the proposed invention represents a short to middle term solution to recipients, for sustaining life, while in waiting lists. Ideally, the same technology as a long-term alternative will be fully tested.

### SUMMARY OF INVENTION

### Cardiovascular Implants

### First Generation

The structure of this implant comprises of a layer of biocompatible matrix that encapsulates the heart in risk of failure (soft matrix based on silicone elastomers and polymers due to their flexibility, adjustability, high compressibility, biocompatibility, and resistance to mechanical forces). There is a built-in set of biocompatible flexible electronic microchips and sensors distributed throughout this soft matrix. Indications found in the Austrian Prio A60273/2019. This matrix is designed using either industrial or publicly license free image analyses software, to extract morphological characteristics from CT/MRI images and to create a structure that adjusts to key morphological points, posing no further risks to the local bio-environment (FIG. 1). In severe cases of mechanical impairment affecting heart rate, an inner membrane can be placed to mechanically expand and contract, assuring that a reasonable level of blood flow rate is preserved.

The sensors and microchips coupled with the soft matrix are dynamically controlled via AI technology. The nano-sensors send monitoring signals to the AI platform, which reacts accordingly, to maintain local homeostasis at acceptable levels. In response to the analysed signals, the AI system triggers electrical and mechanical commands received by microchips (Austrian Prio A60273/2019). These microchips coordinate both electrical impulses transmitted to the heart (example: resynchronization commands in case of left branch block, indicating alteration of the heart's electric conduction) and mechanical commands sent to the soft matrix (example: in case of need for compressive resuscitation due to intense myocardial electric failure).

The resulting structure (soft matrix called “the shell”) is fitted to the target organ using computational superposition and a protocol generated for computer-assisted surgery (robotics)— FIG. 2. For that, the author designed a virtual environment, to support medical experts during prosthetics implantation. This is one of the functionalities found in the core computational system responsible for (i) receive and analyse physiological signals and (ii) store historical physiological signals (Austrian Prio A60273/2019).

Position of sensors and therapeutic components: The integrated components that adopt the system with therapeutic and electric-mechanical properties are placed using the anatomical planes as a reference. Nano-sensors are heterogeneously distributed, in the sagittal plane; with a higher density of nano-components found at the septum and around the four heart valves—mitral, aortic, pulmonary and tricuspid. The locations are set in the indicated manner because ventricular blood flow patterns that give rise to fundamental wall shear-stress and myocardial mechano-elastic forces that drive cardiac function are intrinsically linked to phenomena observed at those locations.

Four groups of model variables: To analyse the physiological signals via AI technology, the variables of interest are divided into groups, according to their utilisation in the computational model. The first group is the Vital Group. Variables in this group are used in mechanical coordination of blood pumping (ECG waves, the cardiac output or blood flow rate measured at the cardiac valves, the heart rate, the heart rhythm, the sinus rhythm, and the myocardial tissue distension and contraction potential as a function of the atrial and ventricular volume using the septum as the geometrical axis of reference). For details on computational fluid dynamics, along with diverse aspects of physiological flow patterns observed throughout the cardiovascular system, please check the following references (Griffith 2005; Stouffer 2008). The second group is the Monitoring Group. Variables in this group indicate physiological trends used to assess supply of nutrients and oxygen to the body. These are blood density/viscosity measured at the cardiac valves, O2 levels, and blood levels of vitamins (e.g., vitamins A at 30-95 mcg/dL and D at 30-60 ng/mL). Other variables can be included in these groups, in future developments. The third group is used to prevent physiological anomalies resulting from implant-host interactions (e.g., immune responses). This group is named Coupling Group and contains variable such as immune cells counting and protein concentration levels (e.g., immunoglobulin levels), coagulant factors' levels (e.g., platelets concentration), and expression markers (e.g., inflammatory chemokines' levels). A fourth group was also defined, which is named Faults Group. This group collects information characterising microelectronics-based functioning, to track the prosthetics functionality, in order to forecast malfunctioning, to trigger action for preventing faults.

Flexible design: The implant can be built in parts according to specific needs. Examples: (1) only resynchronisation and implantable cardioverter-defibrillator sensors and microchips/electrodes placed; (2) only pacemaker placed; (3) the whole composite plus mechanical compressor plus drug-delivery nano-complexes placed. Therefore, this nano-complexes membrane enriched AI-guided cardiac implant varies from conventional cardioverter defibrillators,

cardiac pacemakers, and their variations—e.g. biventricular pacemakers; for being a flexible building blocks structure that grows in complexity according to clinical needs. Therefore, once the anatomic structure is captured, clinical experts can modify the blocks to fit needs identified on the basis of the patient's medical history. This means that the designed implant may start from a single set of micro-fibres (sensors and electrodes) that works as existing cardioverter defibrillators or cardiac pacemakers and be extended, to reach a complete biocompatible soft matrix structure.

### Second Generation

The second generation of cardiac implants contains all the functional variations contemplated in its first generation. The fundamental difference is that now, it would replace the organ in case of irreversible failure. To date, in the medical literature, external devices are used, for a short period, to sustain life during cardiac surgery (organ donation). This is commonly named ventricular assist device (VAD). An external mechanism connects the patient to a heart-lung bypass machine, to keeps oxygenated blood flowing through the body during surgery, ifr the heart stops. Indeed, an implanted left ventricular assist device (LVAD) is the option for waiting for a donor's organ. However, these measures do not endure long-term aid. Therefore, This invention represents a short-to-middle term solution, for severely ill patients in need of heart transplantation (Austrian Prio A60273/2019).

In such case, a denser matrix (3D structural construction using silicone elastomers and polymers) fully coupled to the inertial organ is used, assuring that primary functionality (e.g., movement) is preserved at acceptable levels, in a minimally invasive manner. There are three possible configurations (i) external to the organ to compress and distend, (ii) inflated blocks within heart cavities to expand and contract, or (iii) disconnected to the heart cavity and functioning as an implanted VAD, whose materials and dynamic functionality provided by AI-guided sensors and microchips can endure a longer lifespan.

### Third Generation

The third generation of cardiac implants can be either a soft matrix (first generation) or an organ replacement implanted VAD (second generation). The difference is the addition of the formerly mentioned therapeutic nano-composites and delivery systems. Example: to control the injection of stem cells based therapeutics and to delivery medicinal compounds locally, to restore damaged tissue and local signalling cascades (Austrian Prio A60273/2019). These nano-composites are attached to the implant, at locations prescribed by clinicians, which depends on each patient's case.

Tissue regeneration (stem cells and nanotechnology): Nanotechnology is used in the current medical literature for drug delivery, therapeutic follow-up, and stem cell therapies; minimising surgical invasiveness. Thus, in the proposed prosthetic, the author employs transplanted cells for tissue regeneration. The injected cells might be efficiently fixed and adapt to the target tissue, remaining viable for the necessary lifespan. Therefore, the author proposes collagen-based microcapsules as a doable encapsulation mode for labelled bone marrow—derived mesenchymal cells (MSCs), which are prepared for intra-myocardial injection, guided via MRI monitoring.

Imaging (nanotechnology): Long-term monitoring of stem cells' adhesion and proliferation in regenerative myocardial tissue can be performed via MRI imaging, in presence of nano-contrasts, which are fixed to the implanted cells. In the proposed prosthetic, the author introduces superparamagnetic iron oxide nanoparticles (SPIONs) for labelling implanted stem cells (bone marrow MSCs), because of their magnetic properties, low decay rate, and biocompatibility. As a result, MRI technology can be used to track MSC-based recovery of an infarcted myocardium. Infoiination about diverse research on nanotechnology in biomedicine can be found in (Pereira, 2020, Governing Issues in Nanoscale Systems and Their Potential for Improving the Therapeutic Application of Phytoconstituents, Springer Nature, Plant-derived Bioactives, https://doi.org/10.1007/978-981-15-1761-7_24)

### Additional Features

To enforce safety and autonomy, a set of sensors locally placed would send signals to a monitor in the host patient. These sensors are used to capture signals sent by the implanted sensors, signalling harmful trends using light (either stretchable electronic patches or micro-subcutaneous light cells are used in the alert system). The alert system comprises of three indicative lights: (i) green indicating that the implant is fully functional and that local homeostasis is preserved, (ii) yellow indicating that the sensors are indicating an imminent need for inspection, (iii) red indicating severe risk of failure. This advanced notice would allow time for action to be taken, safeguarding the wellbeing of the host patient. AI orchestrates the whole process. Additionally, the implant communicates with a central unit (hospital computational system and app installed in mobile devices), where clinicians would remotely perform the necessary monitoring (Austrian Prio A60273/2019). The proposed mobile app can be also installed in the patients' devices for self-checking

Similarities with Existing Technology and its Benefits

The technology here proposed regarding materials is well stablished in the pacemakers/VAD industry and in the pharma industry related to nano-composites as delivery systems. Therefore, following regulations stablished by both the World Health Organization (WHO 2003) and worldwide drug and medical devices regulatory agencies, including the American Food and Drug Administration (FDA), the Japanese Pharmaceuticals and Medical Devices Agency (PMDA), the Indian Central Drugs Standard Control Organization (CDSCO), the Chinese agencies deriving from the former State Food and Drug Administration (SFDA), the South African Health Products Regulatory Authority (SAHPRA), the Brazilian National Health Surveillance Agency (ANVISA), the British Medicines and Healthcare products Regulatory Agency (MHRA), and the European Union network of centralised and decentralised agencies throughout its member states; approved cardio-implants technology adds an extra element of safety and reliability to the whole process, while reducing the timescale to have the new model available in the market.

The nano and microscopic dimensions of the implanted components reduce common drawbacks related to surgical invasiveness, post-implantation bleeding, and immune reactions. This is in-line with the most innovative technology employed in existing cardio-implants manufacture.

The biocompatible microchips, silicone elastomers and polymers based biomedical implantable materials here proposed are already commonly used in varied medical applications and are proven to endure physiological challenges appropriately, with a lifetime that commonly spans over 5 to 7 years and even more, without defects.

The core technologies here employed are well known. Therefore, training clinical experts on this new variation of implants that are compatible with existing technologies would not be challenging, because the foundations are well stablished and broadly employed in clinics. This would facilitate the integration of the proposed prototypes into clinics, benefiting patients whose medical history would imply an extra level of aid, better using a composite soft matrix with multiple functionalities and advanced monitoring.

The parts used to build the physical components (e.g., the microcontroller) are already FDA regulated, to be employed in building up medical devices.

### Major Novelty and its Benefits

A single structure can now grow in complexity, keeping the advantages of existing components, which are now integrated in a flexible manner, as indicated in paragraph 0024.

The system is empowered by AI technology, with potentially four groups of variables (signals) collected and transmitted by sensors, which are used to: (i) monitor and adjust device behaviour according to patients' local physiological responses; (ii) forecast critical episodes, alerting on need of medical attention; (iii) dynamically identify threat related to devices' malfunctioning rather than patient's physiology, alone; and (iv) emulate GPS-like location-tracking, at a finer scale, to indicate which parts require repair. Altogether, this can potentially extend the devices' lifetime and increase reliability in real-time monitoring. This, on top of a complete computational platform, providing personalised (CT/MRI based) implant design and guidance for surgical placement, real-time aid and monitoring, along with collection of data for model improvement. FIG. 3 shows different stages of prosthetics implantation. The figure indicates the composite nature of the shell and the varied levels of complexity, which result from coupling a number of small minimally invasive surgical steps. FIGS. 4, 5, 6, and 7 show the components of the implants and their operational modes, along with the results of tests on the AI-based control platform.

The structure proposed includes extra sensors and actuators, plus delivery systems, which results in both assuring fundamental heart function (which conventionally relies on pacemakers, resynchronisers and defibrillators) and myocardial tissue repair whether necessary (on the basis of nano-complexes).

Ultimately, the final level of complexity contemplated in the first generation of cardioprosthetics designs employs mechanical forces driven by pressure. This, in cases where myocardial cells stop responding to electrical stimulus, which requires alternative approaches to prevent irreversible cardiac arrest. Please, notice that single units comprising of pacemakers and defibrillators are intended to be long-term implants. To date, the higher the complexity, the weaker the native cardiovascular system would be. This implicitly means that patients requiring highly complex prosthetics are likely to be in a waiting list for organ transplantation. As indicated in former paragraphs, these are currently the most indicated candidates for “the shell” as a complete unit including the mechanical compressor for resuscitation.

The cardiac implants communicate with computational platforms installed in hospitals, where cardiologists have 24/7 access to monitoring signals. The inventor designed the same level of monitoring automation found in existing cardio-devices (e.g., pacemakers), additionally informing the patient on safety states. The idea is to assure that the patients themselves are also informed 24/7, being able to seek for medical assistance, if a warning signal is tracked. This data can also be transmitted to a mobile app installed in the patient's mobile device. The alerting system would be easy to understand and might not affect the daily routine of the patients, comprising of a subcutaneous microscopic implant, working as a signal receptor, which would translated the received signal (as described in paragraph) into three light-based alerts (green—normal functioning; yellow—medical assistance needed; and red—imminent risk of failure).

Technical innovative designs in process of gaining FDA approval can run in parallel, while fully tested and approved components are launched in the market, improving existing medical procedures.

Replacing, repairing and implanting the proposed prosthetics are minimally invasive, due to both the dimension of the designed components and the fact that the structure is precisely implanted in blocks, via computer-assisted surgery. The proposed technology is designed to make it simple, reliable, and safe; besides the inherent risks of posed by any medical intervention of this scale.

The computational system used to test the AI-model that controls the implants comprises of a 16 cores windows 10 machine, with 32 GB of RAM. The final model is deployed to a microcontroller, which is integrated to the implanted prosthetics. The advantage is that the model can be deployed to numerous microchips (e.g., the nRF52 Series of System-on-Chip (SoC), ESP8266, STM32, and etcetera), resulting in a number of options regarding microcontrollers' technology. This AI model comprises of a robust convolutional neural network (FIG. 6) with batch normalisation and exponential linear units, using a cropped training strategy, which was implemented in python (Anaconda). It was trained on the basis of thousands of historical records and synthetic data was employed to assess the bias-variance trade-off. Multi-classification on tuples (input signals, target action mode) was performed using a new subset of the original dataset (the validation set). As expected, increasing the number of records in the training set resulted in higher accuracy, once the net was pruned for reducing computational complexity; which confirmed that the net generalises well on the validation set (FIG. 7). Indeed, convergence was achieved on a sampling of 75,000 records, with accuracy of c. 97%. These numbers strongly suggest that the model is reliable for clinical application.

### Renal Prosthetics

### First Generation

The first generation of renal implants comprises of a simple set of sensors and micro actuators, which are connected to a control system (microchip). Biomaterials and biocompatible microchips are the ones indicated in paragraphs 0019 to 0043, when referring to microelectronics and biocompatible materials used in the heart implants. However, here, such materials and microelectronics are used for capture physiological signals and measurements (e.g., real time dosage of creatinine in both urine and blood, real time dosage of acid uric in both blood and urine, monitoring of inflammatory biomarkers, and calculation of nephrons' filtering capacity). The AI-control system forecasts renal failure using these variables and responds to imminent threats by sending signals to the actuators, to maintain local homeostasis at acceptable levels (Austrian Prio A60273/2019).

The purpose of this implant is to coordinate (i) real-time renal function monitoring, (ii) drug delivery, (iii) imaging, and (iii) regenerative tissue therapeutics (e.g., based on stem cells technology) in patient with reduced renal capacity, without the indication of nephrectomy. Therefore, the diseased organ is constantly monitored and treated (Austrian Prio A60273/2019).

### Second Generation

Again, all the assumptions on biocompatible materials, microcontrollers, and sensors formerly set apply to the second generation of renal implants. However, the region of interest changes, as kidney implants are the targets, instead of artificial myocardial tissue (Austrian Prio A60273/2019.

While the first generation of renal prosthetics (e.g., the cardiac implanted prosthetics discussed in the former section) here presented (paragraphs 0044 and 0045) is intended to correct local impairment, without replacing an organ; this second generation of prosthetics work as a fully functional artificial kidney, following nephrectomy (Austrian Prio A60273/2019). This would leverage the workflow on one single kidney.

The implant is a 3D printed reconstructed structure replicating the patient kidney's target volume, whose design is personalised, according to 3D reconstructed and segmented CT/MRI data (FIG. 8). However, the implant is not required to follow anatomic patterns, precisely, but is designed to facilitate filtration and pumping at a target chamber volume. The manufactured artificial organ is fitted to the target location via the same virtual environment shown in FIG. 2.

To adopt the system with important functional capabilities, if surrounding impaired tissue or local homeostasis need to be treated, the therapeutic components (e.g., drug delivery and stem cells technology) indicated in paragraphs 0019 to 0043 are used.

Again, sensors are strategically placed to monitor the prosthetics functionality for mitigating faults, measure physiological flow drivers (e.g., gradient of pressure) and concentration of blood compounds (e.g., concentration of dialysed uric acid) to control filtering and pumping mechanisms, along with concentration of chemical compounds leading to physiological impairment such as hypocalcaemia, to feedback safety and alert mechanisms (FIG. 9). FIG. 10 shows the invention here presented (implantable artificial kidney).

Four groups of model variables: Here, the variables of interest are equally clustered in groups, according to their utilisation in the model. Please, notice that groups' names are standardised for usage with all the existing prototypes. In the Vital Group one finds variables used in filtering and mechanical coordination of haemodialysis. These variables are the pressure at both the renal artery and renal vein, the volume of blood inflow as the filtering chamber controls inflows leading to a maximum volume (e.g., 500 millilitres), the concentration of reabsorbed compounds (such as water, sodium, bicarbonate, glucose, and amino acids) before and after diffusion through the porous membranes, and the concentration of dialysed molecules (such as hydrogen, ammonium, potassium and uric acid) before and after diffusion, among others. For details on computational fluid dynamics, along with diverse aspects of renal function and glomerular filtration, please check the following references (Jo, Ryu, Kim, Lee, & Choi, 2019). The Monitoring Group variables here define physiological trends used to assess whether homeostasis is properly preserved in the urinary and lymphatic (renal lymphatic) systems. These are the concentrations of reabsorbed blood compounds. Again, other variables can be included in these groups, in future development. As formerly indicated, the Coupling Group is used to prevent physiological anomalies resulting from implant-host interactions, which are mostly detected via impaired immune responses. This group contains the same variables for all the implanted models. Finally, the Faults Group defines microelectronics-based functioning, tracking prosthetics functionality, to mitigate faults.

### Third Generation

The third generation of renal implants, simply imply bilateral nephrectomy and the use of implants for complete renal function.

### Additional Features

As in paragraph 0030, additional features relate to safety light signals based mechanisms and complete remote signals transmission from implants' sensors/microchips to external mobile devices and computers.

### Similarities with Existing Technology and its Benefits

The fundamental point is that the biocompatible materials and microchips used are well-stablished in the biomedical industry, having passed the usual standards checks for safety and quality assurance. This safeguards the material construction of the invention, regarding reliability and predictable lifespan. This, along with all the relevant repurposing points indicated in paragraphs 0031 to 0035.

### Major Novelty and its Benefits

The first generation of kidney implants dynamically couples varied therapeutic elements, coordinating via AI technology both real time monitoring and real time drug/therapeutics delivery to damaged tissue, remotely (microchips for GPS and wireless connection) informing clinical teams and the patient about both health condition and functional state of the implant.

The second generation of nano sensors-based AI-modulated kidney implants replaces an entire organ, following nephrectomy, to leverage overloading of the renal system.

In cases of bilateral nephrectomy, where organ transplantation is still a long way ahead, the designed third generation of implants is an alternative to existing haemodialysis devices. Haemodialysis is currently undertaken by either large machines placed in clinics or automated units for homecare. In the process, the patient's blood is exposed to the local environment, leading to risk of contamination by bacteria and other pathogens, which can result in satellite diseases, because even the best aseptic practices may still do not totally prevent infection. Therefore, this implanted devices would reduce underlying risks of conventional haemodialysis devices.

Clearly, haemodialysis has allowed patients suffering from end kidney diseases to survive an average of 10 years and more. However, envisioning a portable device, which is here translated into biocompatible implanted prosthetics, can leverage the pain and discomfort that haemodialysis patients are subject to, daily basis. Indeed, it worth mentioning that psychological factors play an important role in the well-being of individuals suffering from pathologies that severely compromise their daily routine. Indeed, this may be even more significant when treating infants. Therefore, this invention (implanted mechanisms) can radically change the perspectives for haemodialysis patients. Additionally, as previously indicated, implanted mechanisms would reduce exposure to infectious agents and potentially allow bilateral nephrectomy patients to live normally, without the need of a kidney transplant, for much longer than what is predicted when using existing haemodialysis devices.

The challenge in manufacturing implanted units is on the current large volume of dialysis fluid required in the majority (if not all) of effective protocols. To overcome this drawback, this invention is designed on a novel chamber mechanism that filters the blood (osmosis based as in conventional haemodialysis) using multiple filters, according to the size of the target blood compound. This new chamber does not require the use of haemodialysis liquid, because it calculates the water disposal volume per haemodialysis cycle, using the excess to produce artificial urine (FIG. 10)

In the kidneys of healthy adults, a system leaded by nephrons and glomerular filtration receives around 1 litre of blood per minute, in average (about 20% of the heart output), and excrete c. 1.5 litres of urine per day. The artificial chamber here indicated is capable of maintaining acceptable levels of homeostasis, with c. 500 millilitres of blood being filtered in each chamber, per cycle. This reference volume is set via initial 3D design, as indicated in former paragraphs.

Physiological mechanisms pose severe challenges to synthetic biology. In biocompatible implanted prosthetics design, the labour is not easier. Extrinsic mechanisms underlying renal function relate to both neural and hormonal control that override renal autoregulation. This is intended to safeguard the living body when unexpected phenomena occur. Example: although urine is produced at a certain level, even when an individual is suffering from dehydration, to assure that blood is freed from toxins, haemorrhage leading to severe decrease in blood pressure would cause reduction in urine production, in a process modulated by the neuronal system not by renal autoregulation, alone. This invention simplifies real physiology, though preserving fundamental features. Therefore, this invention mimics urine production, in a haemodialysis fashion, where novelty is in the absence of haemodialysis fluid (commonly found at 1.5% dextrose dialysis solution, in 5000 ml flasks) and in the proposed implanted mechanisms coordinated by AI technology and adopted with therapeutic and drug delivery elements.

Key functionality is found in the dynamic control of mechanical processes, as indicated in FIG. 10. Additionally, timing for filtering phases is fundamental. Furthermore, signals transmitted by sensors, to feed both monitoring and action systems are equally part of the dynamic operations that are here orchestrated by AI technology.

A gradient boost model was built and trained, running on a 16 cores windows 10 machine, with 32 GB of RAM. Again, the final model is deployed to a microcontroller, which is integrated to the implanted prosthetics. The performance of this GBoost model here presented is illustrated in FIG. 11. This AI model was trained on the basis of thousands of historical records and synthetic data was employed to assess the bias-variance trade-off. Forecasting relied on tuples (input signals, target urine production) for model validation, on a new subset of the original dataset (the validation set). It is noticeable that model accuracy increases direct proportionally to sampling size and model generalisation seems to be accord with expectations, if compared with predictive models found in the current literature. To the best of the inventor's knowledge, this is the very first time that this specific biophysical problem is approached, in the literature. However, forecasting on physiological datasets is a common application of GBoost algorithms, which sets the criterion on acceptable quality assurance. Model convergence was achieved on a sampling of 10,000 records, with an accuracy of c. 94%. These numbers strongly suggest that the model is reliable for clinical applications.

### Neural Implants

### First Generation

This implant comprises of nano-composites combined with sensors that are controlled by AI technology, for signals analysis, dynamically monitoring and triggering the delivery of chemicals to brain tissue. The inventor designed different models that use similar structures, varying slightly according to both target signalling cascade (or group of neuronal cells to be treated) and needed compound to be delivered (Austrian Prio A60273/2019).

The materials used to encapsulate implanted devices and to form artificial shells (artificial tissues) are the same biocompatible materials indicated in paragraphs 0019 to 0043. Equally, microchips used in sensors for electrical signals registration probes, in imaging, and in AI-based implant's control use the same technology indicated in paragraphs 0019 to 0043. The purpose is to monitor, analyse and control/adjust chemical reactions and the relevant signalling cascades (Austrian Prio A60273/2019).

Model 1: Bio-implant that collects local electrical and biochemical signals and use AI technology to drive immune assays for biomarker determination and knockdown of diseased signalling networks. The target is amyloid and/or tau, modulating diseased signalling cascades, to mitigate Alzheimer plaque build-up (Austrian Prio A60273/2019).

Model 2: Bio-implant for local physiological monitoring, dynamically delivering compounds, to mitigate excitotoxicity caused by imbalance in expression of neurotransmitters. Excitotoxicity deteriorates neuronal tissue, resulting in several existing chronic neurodegenerative processes, including multiple types of dementia and defective working memory. Therefore, this model is used in numerous therapeutics (Austrian Prio A60273/2019).

Model 3: Bio-implant for drug delivery and disease progress follow-up (dynamically capturing signals during treatment, which are analysed in real time, to monitor progress and trigger local drug administration accordingly, in response to different physiological responses). This model is precisely designed for tissue recovery, following for instance brain injury caused by strokes or cranial traumatism resulting from road accidents, and other critical episodes (Austrian Prio A60273/2019).

### Second Generation

The second generation of neuronal implants are signals transmitters leveraging electromagnetic dysfunction. The implants will communicate with an AI platform for signals processing and analysis. To date, there are two target systems, resulting in two designs (Austrian Prio A60273/2019).

Again, the materials used to encapsulate implanted devices and to form artificial shells (artificial tissues) are the same biocompatible materials indicated in paragraphs 0019 to 0043. Equally, microchips used in sensors for electrical signals registration probes, in imaging, and in AI-based implant's control use the same technology indicated in paragraphs 0019 to 0043. The purpose is to monitor, analyse and control/adjust electromagnetic signals that are related to functional impairment like visual dysfunction and hearing loss. Implantable silica fibers, hydrogels (e.g., hydrogels derived from polyethylene glycol diacrylate for slab waveguides), synthetic polymers, and elastomers for sound, optical, electrical and chemical signals' transmission are used (Austrian Prio A60273/2019).

Model 1: Biocompatible electrical encoder-decoder implants to support the transmission of visual information from the retina to the brain (Austrian Prio A60273/2019), when the process is compromised by optical nerve damage, mitigating vision loss (e.g., in patients suffering from glaucoma).

Model 2: Biocompatible electrical encoder-decoder implants to support the transmission of sound from the cochlea to the brain, when the process is compromised by auditory nerve fibres damage, mitigating hearing loss (Austrian Prio A60273/2019).

### Additional Features

As in paragraph 0030, additional features relate to safety light signals based mechanisms and complete remote signals transmission from implants' sensors/microchips to external mobile devices and computers (Austrian Prio A60273/2019).

### Similarities with Existing Technology and its Benefits

Again, the biocompatible materials and microchips used are well-stablished in the biomedical industry, having passed the usual standards checks for safety and quality assurance, resulting in increasing reliability and predictability regarding lifespan. This, along with all the relevant repurposing points indicated in paragraphs 0031 to 0035.

### Major Novelty and its Benefits

It has not yet presented, in the current market, implants that combine three core elements—AI technology, novel biomaterials, and biocompatible microelectronics; in a conjugated manner to measure and resolve both transmission and processing of sound, optical, electrical and chemical signals by the brain. Therefore, while supported and founded by existing and well-stablished technology, this state of the art invention represents one more step towards improving existing brain-related impairment.

### BRIEF DESCRIPTION OF DRAWINGS

FIG. 1. Illustration of shell design. The first step in the manufacture process is to generate the structural models, which can be done via most of the existing segmentation and 3D reconstruction software. The author here indicates the use of Materialise 3-matic, version 13.0. In the diagram, one can see (a) how CT/MRI images are used for matching patients' anatomy and the implanted membrane; (b) how the images are automatically segmented for extracting the regions of interest (ROI); (c) and (d) how remaining artefacts are manually removed and the resulting structure prepared for further filtering; (e), (0, and (g) the final coupling membrane, the removal of the inner part of the solid, the shell thickness being fixed at 1-2 mm, and the final structure further segmented according to the regions of interest; and (h) an illustration of the diverse structural parts that can be manufactured, on the basis of anatomic CT/MRI images. Further filtering can be employed to smooth the superficial layer, removing final artefacts. The final anatomic models are saved in stl format for further use.

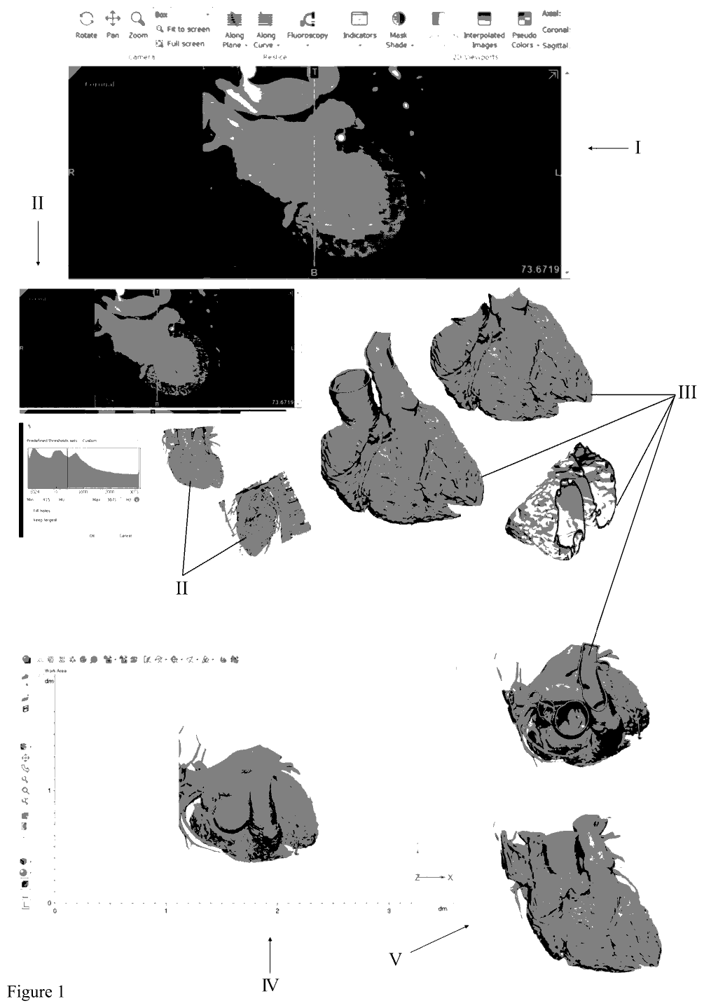

FIG. 2. Illustration of the coupling shell-organ. Diagrams a and b illustrate normal and impaired diastole-systole, respectively. Indeed, illustrations a and b left indicate heart relaxation, while illustrations a and b right illustrate heart muscular contraction. In diagram b right, it can be seen that systole is compromised by reduced ability of the muscle fibres to contract. Diagram c illustrates restoring contraction potential resulting from mechanically driven implant aid. Diagram c also shows a close-up view of the coupling shell-tissue. Illustration d presents the design of the virtual environment to be used by medical experts, in order to perform shell-organ coupling and robotics driven protocol generation, preceding a computer-assisted surgery.

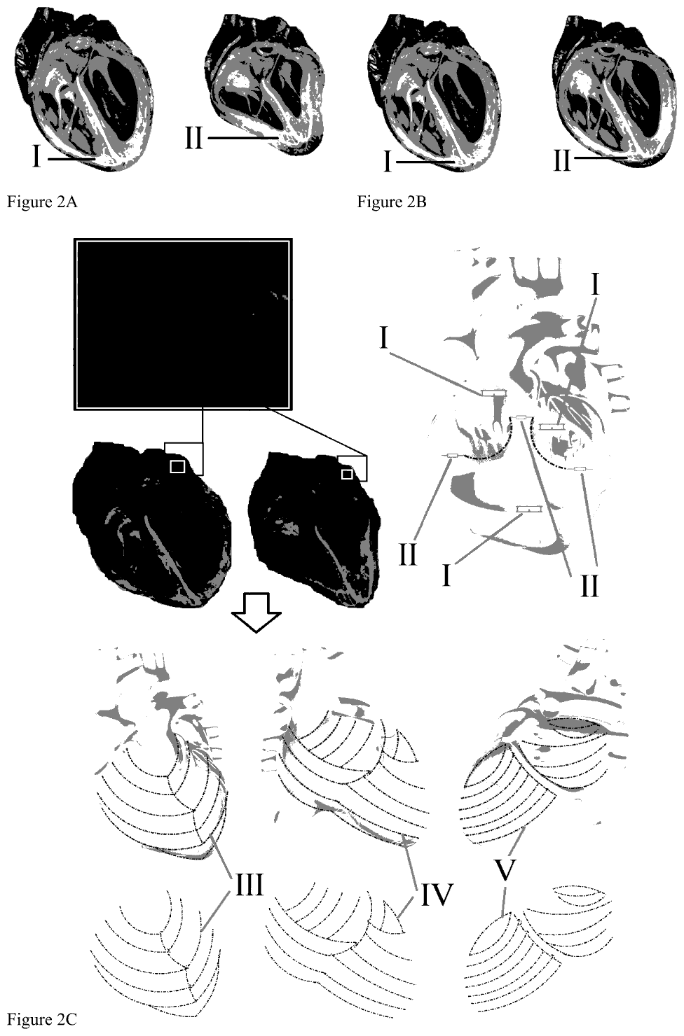

FIG. 3. Illustration of prosthetics implantation. The proposed implanted modulus are shown. Pacemakers, resynchronisers, and CDIs are introduced intra-venous, being implanted in the cardiac muscle, as usual. The mechanical pacemaker here presented (mechanical compression in case of severe electrical failure) is designed to form a shell (soft matrix) and is introduced externally, to apply contractile forces on the heart chambers. However, in severe cases, where myocardial stiffness increases abnormally and the cardiac valves become defective, implanted modules are introduced within the heart chambers, alternatively, either inflated within the chambers (flexible silicone-based membranes) functioning as inner flexible pumps or fixed to the heart valves to facilitate their movement.

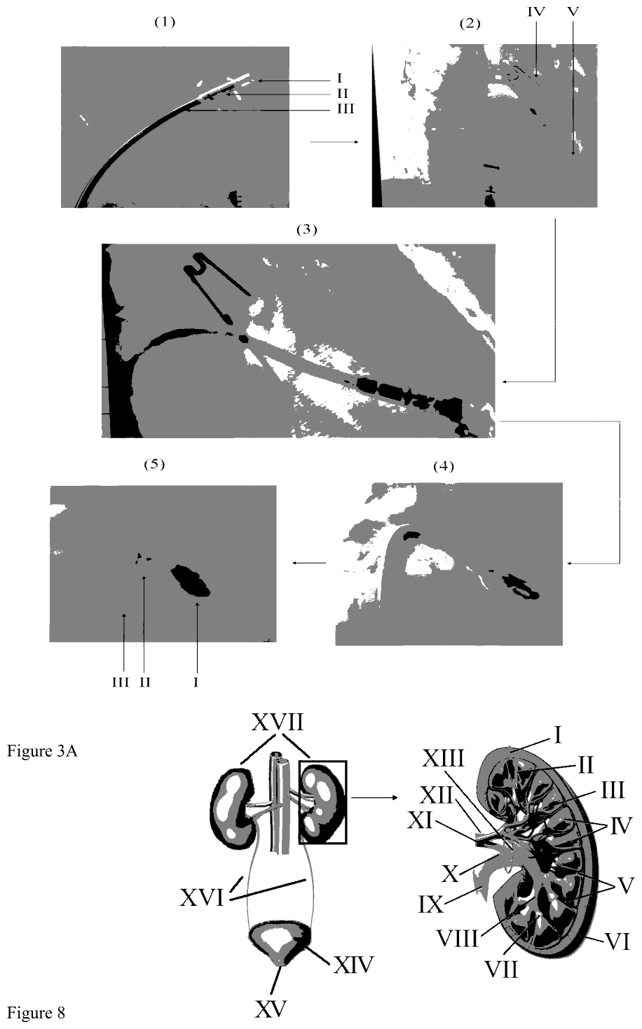

FIG. 4. Schematic view of the prosthetic components and operational modes: (a) illustration of groups of components; (b) operational modes; and (c) security and functional management. This figure shows a simplified overview of varied operational modes found in the model: (i) pacemaker, (ii) defibrillator, and (iii) mechanical compressor. These operational modes rely on both physical components and mathematical abstraction translated into algorithms implemented via computational pieces of software. Regarding physical components, three major groups of components were designed: (i) sensors, (ii) actuators, and (iii) optional components as nano-complexes for imaging, drug delivery, and stem cells therapy. The group of optional components are classified as biodegradable and resident, according to the respective medical prescription. Finally, three components are used for safety and functional management. These are a programmed micro-chip (AI microcontroller) that performs AI-driven decision making; a micro light-monitor that alerts on critical conditions using green, yellow, and red lights; and a computational platform that is placed remotely (e.g., in hospitals and clinics) operating as a General Control Prosthetics (GCP) platform. The GCP platform also hosts an advanced version of the AI-model named backtrack-convnet, which is coupled with a reinforcement learning algorithm, in an adversarial network architecture, which receives signals from the implanted sensors and dynamically interacts with the backtrack-convnet, to update it dynamically, giving raise to its new versions, in order to periodically update the AI microcontroller.

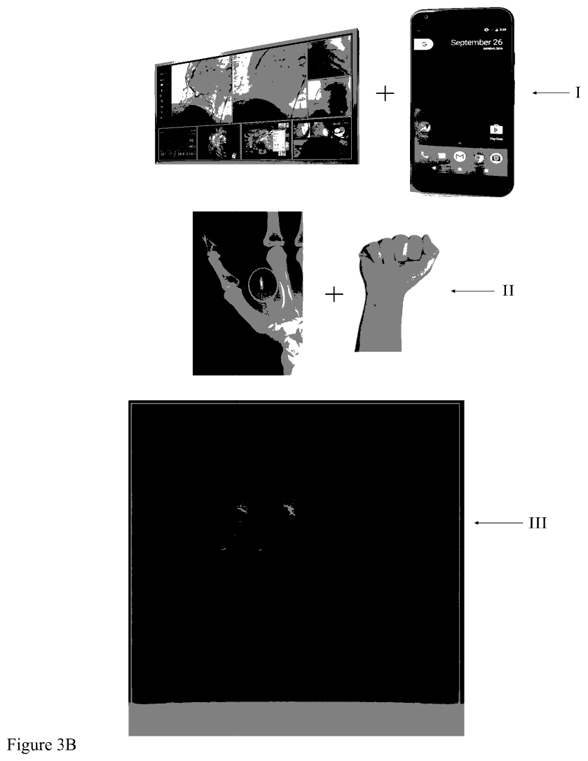

FIG. 5. Illustration of functional abstraction (data transmission and GCP platform): (a) sensors transmit signals to the AI microcontroller, which are analysed and translated into commands that are sent to the actuators; (b) signals are also sent by the sensors to both the light monitor for security alerts and to the GCP platform for clinical monitoring and AI model improvement, using newly collected data; and (c) clinical monitoring is performed remotely, on the GCP platform, and in the same monitoring system, generation of AI model updates takes place, using a newly created backtrack-convnet in an adversarial network architecture.

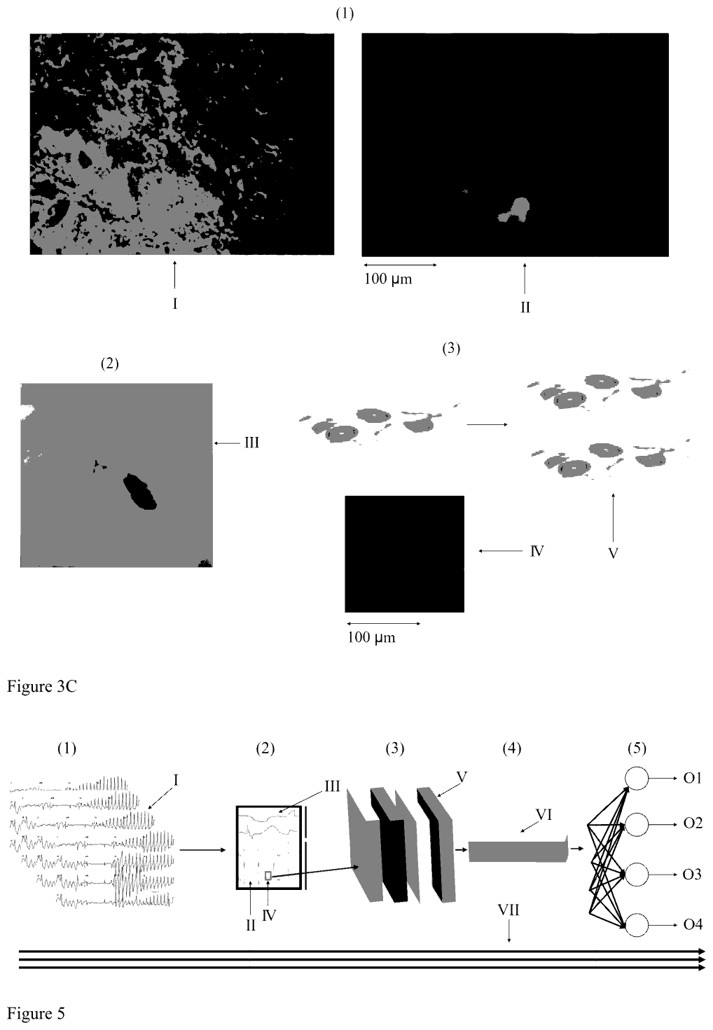

FIG. 6. Schematic view of the AI model architecture. The AI model comprises of a robust convolutional neural network with batch normalisation and exponential linear units, using a cropped training strategy implemented in python (Anaconda). It was trained on the basis of thousands of historical records and synthetic data was employed to assess the bias-variance trade-off. Multi-classification on tuples (input signals, target action mode) was performed using a new subset of the original dataset (the validation set).

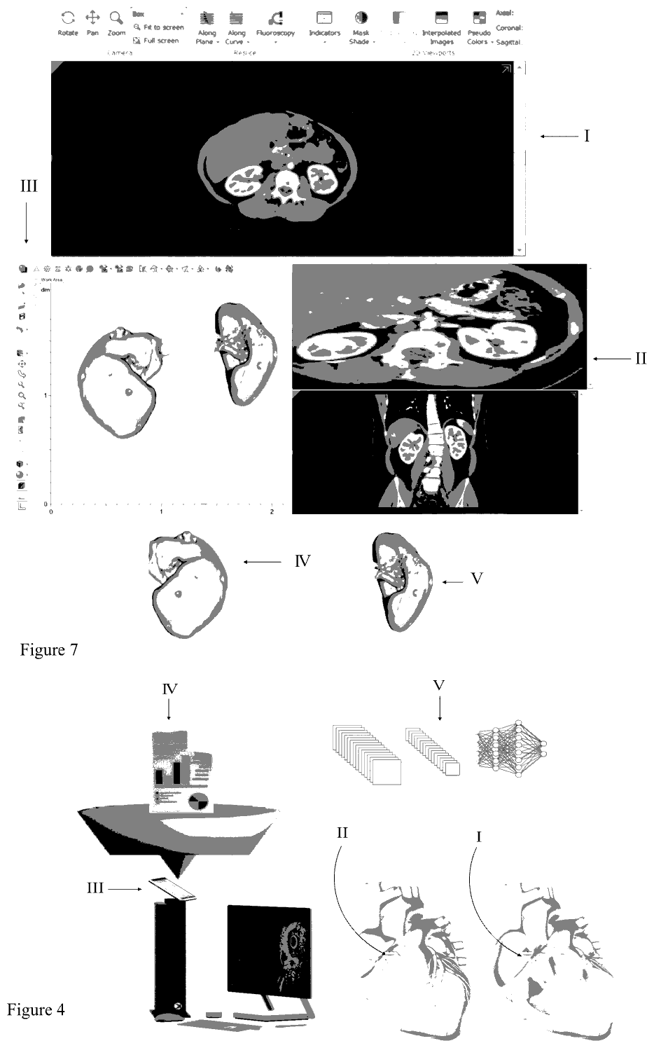

FIG. 7. Results showing ConvNet's performance selecting action mode during myocardial dysfunction. Quality assurance is indicated in diagrams (a) and (b), which show the learning curves as a function of the epochs and batches, along with sampling size, respectively. Diagram (c) shows the model generalisation power and diagram (d) illustrates model application. Given the results, high volumes of data can be collected over a reasonable period—i.e. about 13 days, assuming that measurements follow the physiological interval between consecutive heartbeats, which is 1.1 seconds in average; in candidates for receiving these implants. This would empower the AI microcontroller with dynamic self-calibration, resulting in model re-training to assure consistency and dynamic self-adaptation to changing conditions (sampling variance), following surgery. Therefore, the initial trained model can adapt to changing conditions, on the basis of continuous data collection and monitoring.

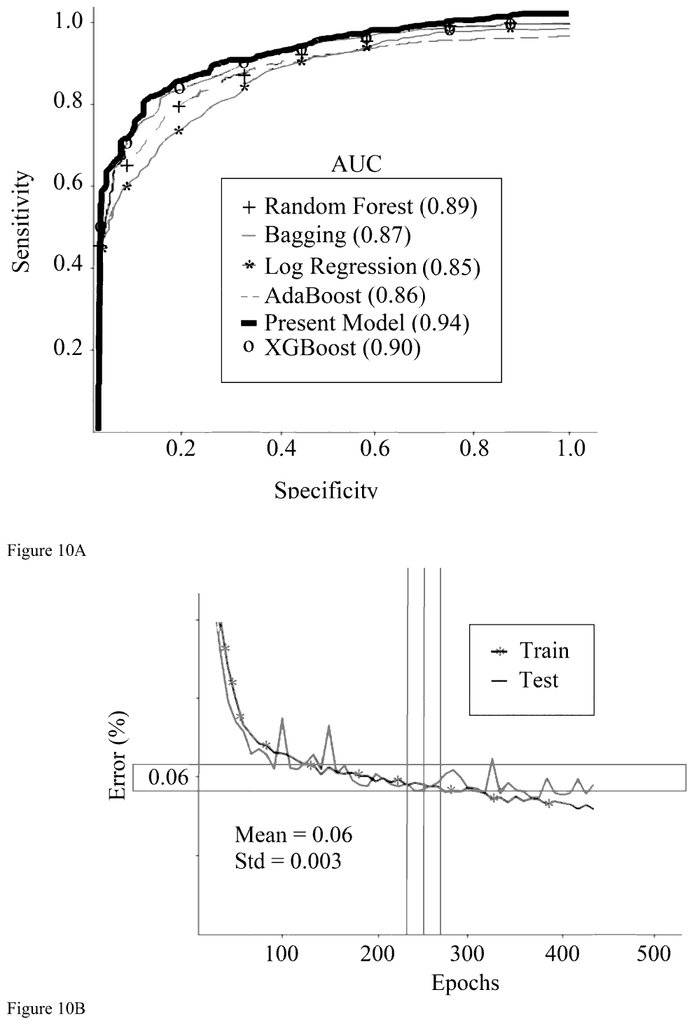

FIG. 8. Illustration of shell design showing complete kidney replacement: (a) and (b) CT scan segments; (c) 3D reconstruction of left and right kidneys; (d) left and rights kidneys' volume extraction and imprint of sensors placement, to be followed in the artificial 3D printed prototype.

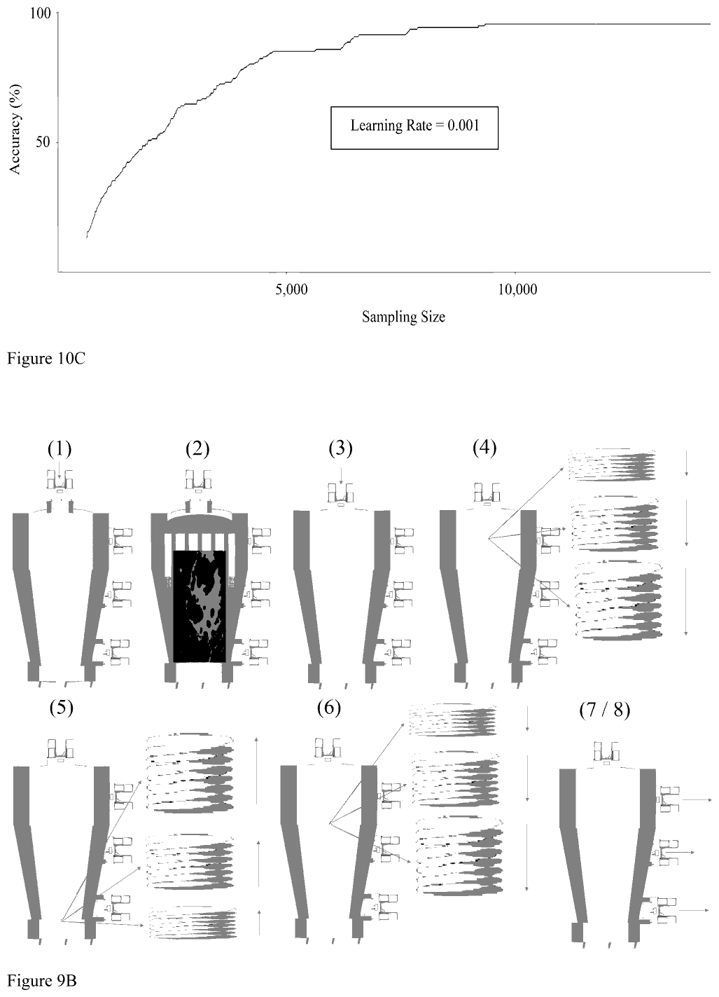

FIG. 9. Graphical summary of the kidney morphology, which defines the characteristics of the prosthetics; and illustration of sensors placement, which correlates with imprints determined via 3D anatomical reconstruction.

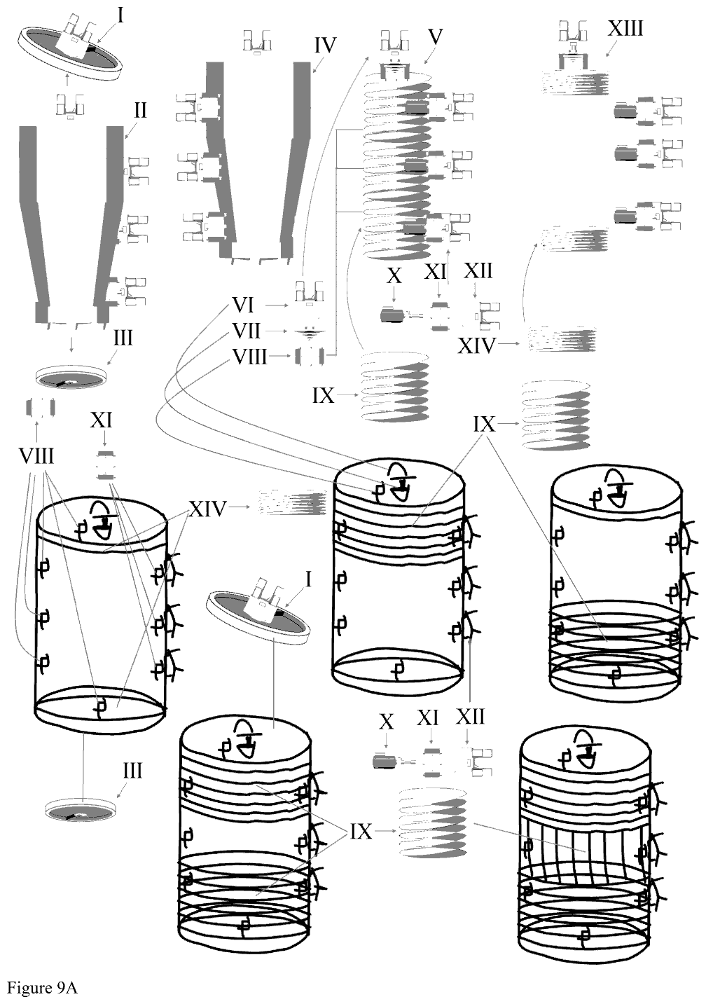

FIG. 10. Schematic view of the artificial chamber designed to filter blood. Diagram (a) shows the major chamber parts—the shell, the renal artery connection valve, the upper and the lower waste cavities, the middle clean blood cavity, the ureters connection valves, the renal vein connection valve, the volume markers and the filters simulating the three main glomerular capillary structures involved in blood filtering (the endothelial pores of 70 to 100 nanometres in diameter, the basement membrane region, and the epithelial podocytes area). Diagrams (b) and (c) indicate the envisioned haemodialysis cycle, which comprises of 8 steps. First, renal artery connector opens. Second, the chamber fills in. Third, renal artery connector closes. Fourth, middle particles (predominantly) filtering occur in the upper waste cavity. Fifth, essential particles remain in the middle clean blood cavity and small particles are collected in the lower waste cavity. Sixty, the volume markers close all cavities. Seventh, clean blood in the middle cavity is pumped through the renal vein connection and waste solution (artificial urine) stored in both the upper and the lower cavities are pumped through the ureters' connections. Eighth, all the volume markers open, finishing the cycle, allowing the renal artery connection to open, once more, and the process to continue.

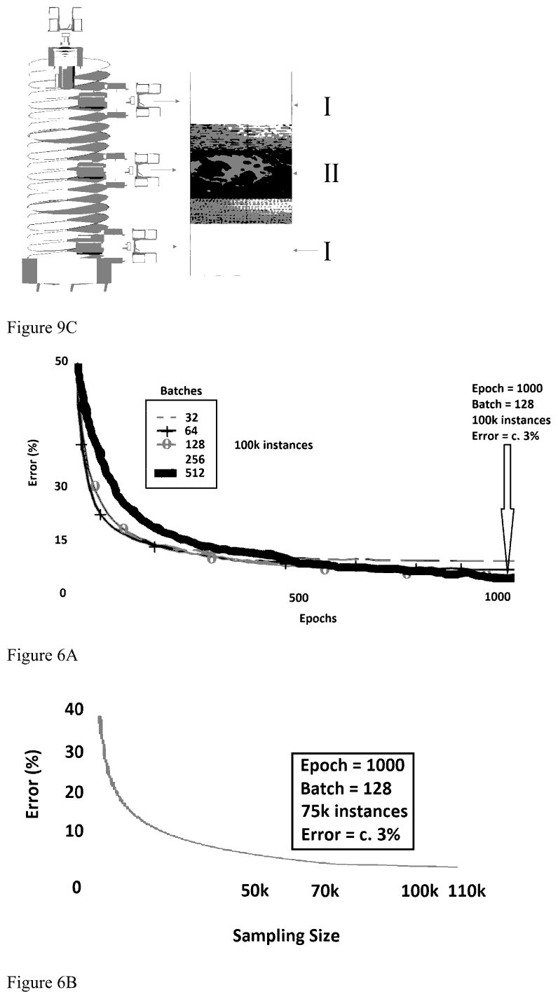

FIG. 11. Results showing Gboost-based model's performance managing prosthetics operation, on the basis of filtration time as a function of target molecules concentration and physiological condition. Quality assurance is indicated in diagrams (a) and (b), which show comparison with other strategies (ROC curves) and the error (3-fold avegare) vs. epochs for early stop, respectively. Diagram (c) shows model performance as a function of sampling size.

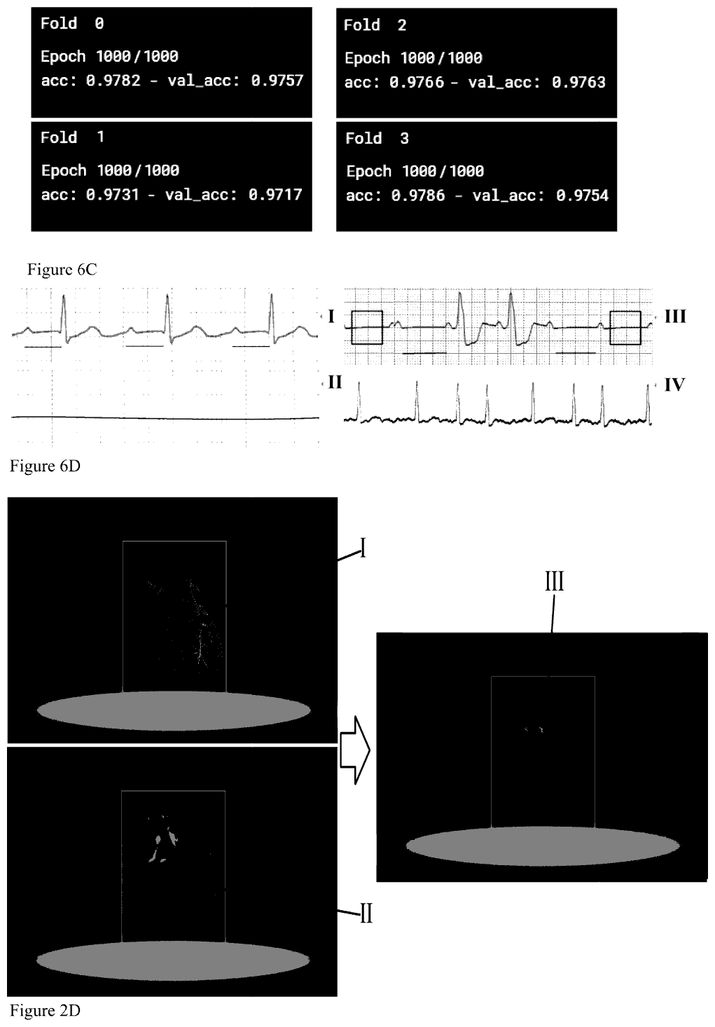

### BIBLIOGRAPHY

Moglia A, Menciassi A, Dario P, Cuschieri A (2009) Capsule endoscopy: Progress update and challenges ahead. Nat. Rev. Gastroenterol. Hepatol. 6:353-361
Canales A, Jia X, Froriep U P, et al (2015) Multifunctional fibers for simultaneous optical, electrical and chemical interrogation of neural circuits in vivo. Nat Biotechnol 33:277-284. doi: 10.1038/nbt.3093
Pereira G (2017a) Genomics and Artificial Intelligence Working Together in Drug Discovery and Repositioning: The Advent of Adaptive Pharmacogenomics in Glioblastoma and Chronic Arterial Inflammation Therapies. In: Malik S (ed) Biotechnology and Production of Anti-Cancer Compounds. Springer-Nature, p 30
Pereira G C (2019) Application of Biotechnology in Producing Plant Bio-active Compounds. In: Natural Bio-active Compounds. Springer Singapore, pp 59-78
Pereira G C, Malik S, Kis Z, Rocamonde B (2019) Computationally Designed Recombinant-DNABased Compounds Production Driven in Plants During Secondary Metabolism and Their Implication in Antimalarial Therapies. In: Natural Bio-active Compounds. Springer Singapore, Singapore, pp 127-146
Boutry C M, Beker L, Kaizawa Y, et al (2019) Biodegradable and flexible arterial-pulse sensor for the wireless monitoring of blood flow. Nat Biomed Eng 3:47-57. doi: 10.1038/s41551-018-0336-5
Shin J, Yan Y, Bai W, et al (2019) Bioresorbable pressure sensors protected with thermally grown silicon dioxide for the monitoring of chronic diseases and healing processes. Nat Biomed Eng 3:37-46. doi: 10.1038/s41551-018-0300-4
Seignez C, Phillipson M (2017) Implanted biomaterials: Neutrophil-mediated vascularization. Nat. Biomed. Eng. 1
Bank R A (2019) Limiting biomaterial fibrosis. Nat. Mater. 18:781 Bannerman D, Pascual-Gil S, Radisic M (2019) An optimal gel patch for the injured heart. Nat Biomed Eng 3:592-593. doi: 10.1038/s41551-019-0440-1
Liu Y, Liu J, Chen S, et al (2019b) Soft and elastic hydrogel-based microelectronics for localized low-voltage neuromodulation. Nat Biomed Eng 3:58-68. doi: 10.1038/s41551-018-0335-6
Conte R, Marturano V, Peluso G, et al (2017) Recent Advances in Nanoparticle-Mediated Delivery of Anti-Inflammatory Phytocompounds. Int J Mol Sci 18:709. doi: 10.3390/ijms18040709
Singh M, Devi S, Rana V S, et al (2019) Delivery of phytochemicals by liposome cargos: recent progress, challenges and opportunities. J Microencapsul 36:215-235. doi: 10.1080/02652048.2019.1617361
Pereira G C (2020c) Governing issues in nanoscale systems and their potential for improving the therapeutic application of phytoconstituents. In: Swamy M K, Saeed M (eds) Plant-derived Bioactives—Chemistry and Mode of Action. Springer Nature Liu T, Liu S, Tu K H, et al (2019a) Crested two-dimensional transistors. Nat. Nanotechnol. 14:223-226
Masvidal-Codina E, Illa X, Dasilva M, et al (2019) High-resolution mapping of infraslow cortical brain activity enabled by graphene microtransistors. Nat Mater 18:280-288. doi: 10.1038/s41563-018-0249-4
Tite T, Chiticaru E A, Burns J S, Ionitâ M (2019) Impact of nano-morphology, lattice defects and conductivity on the performance of graphene based electrochemical biosensors. J. Nanobiotechnology 17
Liu H, Zhao T, Jiang W, et al (2015) Flexible Battery-Less Bioelectronic Implants: Wireless Powering and Manipulation by Near-Infrared Light. Adv Funct Mater 25:7071-7079. doi:10.1002/adfm.201502752
Anumanchipalli G K, Chartier J, Chang E F (2019) Speech synthesis from neural decoding of spoken sentences. Nature 568:493-498. doi: 10.1038/s41586-019-1119-1
Akbari H, Khalighinejad B, Herrero J L, et al (2019) Towards reconstructing intelligible speech from the human auditory cortex. Sci Rep 9: doi: 10.1038/s41598-018-37359-z
Gholami B, Haddad W M, Bailey J M (2018) AI in the ICU: In the intensive care unit, artificial intelligence can keep watch, IEEE Spectr 55:31-35. doi:10.1109/MSPEC.2018.8482421
Pereira G (2020a) Computational Approaches in Drug Development and Phytocompounds Analysis. In: Swamy M K, Saeed M (eds) Plant-derived Bioactives—Chemistry and Mode of Action. Springer Nature
Kumar S N, Lenin Fred A, Ajay Kumar H, et al (2020) Segmentation of Anomalies in Abdomen C T Images by Convolution Neural Network and Classification by Fuzzy Support Vector Machine. pp 157-196
Nagarajan G, Sathish Kumar B S (2020) Proficient Reconstruction Algorithms for Low-Dose X-Ray Tomography. pp 237-256
Pereira G C (2017b) Genomics and Artificial Intelligence Working Together in Drug Discovery and Repositioning: The Advent of Adaptive Pharmacogenomics in Glioblastoma and Chronic Arterial Inflammation Therapies. In: Biotechnology and Production of Anti-Cancer Compounds. Springer International Publishing, Cham, pp 253-281
Pereira G (2020b) Polyphenols role in autoimmune and chronic inflammatory diseases and the advent of computer-driven plant therapies. In: Swamy M K, Saeed M (eds) Plant-derived Bioactives—Chemistry and Mode of Action. Springer Nature
NHSBT (2017) NHSBT Organ Donation and Transplantation: Activity Figures for the UK-2017 statistical report
WHO (2019) WHO|Outcomes of organ transplantation. https://www.whoint/transplantation/gkt/statistics/kidney_outcomes/en/. Accessed 16 Dec. 2019
NHSBT (2019a)NON-PROCEEDING DECEASED DONORS NHSBT (2019b) Organ Donation and Transplantation Statista (2019) Patient deaths on organ transplant waiting list 2019 Statista. https://www.statista.com/statistics/519829/patient-deaths-on-organ-transplant-waiting-listunited-kingdom-uk/. Accessed 16 Dec. 2019
NKF (2016) Organ Donation and Transplantation Statistics 1National Kidney Foundation. https://www.kidney.org/news/newsroom/factsheets/Organ-Donation-and-Transplantation-Stats. Accessed 16 Dec. 2019
Health Resources & Services Administration (2019) Organ Donation Statistics Organ Donor. https://www.organdonor.gov/statistics-stories/statistics.html. Accessed 16 Dec. 2019
Organ Donor Foundation (2016) Organ Donor Foundation—Statistics. https://www.odf.org.za/infoand-faq-s/statistics.html. Accessed 16 Dec. 2019
Chan-on C, Sarwal M M (2017) A comprehensive analysis of the current status and unmet needs in kidney transplantation in Southeast Asia. Front. Med. 4
Stouffer G A (2008) Cardiovascular Hemodynamics for the Clinician. Blackwell Publishing Ltd.

1. Heart Implant (1st generation): A soft biocompatible membrane mimicking the heart anatomy, which the inventor here calls biocompatible matrix or “the shell”. Manufacturing, biocompatible materials and microchips are indicated in the Description. This shell contains microprobes and electrodes to work as conventional heart implants—e.g., pacemakers, CDIs, and resynchronisers. This shell applies mechanical forces on the heart in cases of severe heart electric failure (resuscitation). This shell is coupled with sensors for monitoring vital signals and with microchips-based actuators that acts on the heart. This shell is coupled with an AI-driven microcontroller that receives signals from sensors, analyses these signals, sends action signals to microchips-based actuators, and coordinates/controls the whole system. The shell, in severe cases of mechanical impairment affecting heart rate, is either coupled or replaced by an additional inner membrane (shell) placed to mechanically expand and contract inside the heart chambers, assuring that a reasonable level of blood flow rate is preserved. This inner shell also corrects the heart valves' movement, if needed. Software for AI-driven implant control is installed in a device similar to a pacemaker, which is superficially implanted under the skin, as usual—AI microcontroller).

2. The shell according to claim 1 has a flexible design and can be built in parts—e.g. (1) only resynchronisation and implantable cardioverter-defibrillator sensors and microchips/electrodes placed; (2) only pacemaker placed; (3) the whole composite plus mechanical compressor plus drug-delivery nano-complexes placed. Different from conventional pacemakers, CDIs, and resynchronisers, the shell possesses all these functionalities plus the application of mechanical forces whether electrical impulses fade. This shell is also structurally distinct from conventional heart implants, because (i) it is a shell that covers the outer cardiac structure and (ii) a second shell layer can be placed in the inner heart to improve aid.

3. Heart Implant (2nd generation): A denser matrix (3D structural construction using silicone elastomers and polymers). There are three possible configurations for this shell, which apply according to disease severity and clinical indication: (i) external and covering the organ to compress and distend, according to claim 1; (ii) inflated blocks within heart cavities to expand and contract, according to claim 2; or (iii) disconnected from the heart cavity and functioning as an implanted ventricular assist device (VAD), whose dynamic functionality is provided by AI-guided sensors and microchips, and can endure a longer lifespan.

4. Heart Implant (3rd generation): Either a shell according to claim 1 or a denser matrix VAD according to claim 3 wherein contains additional structures for therapeutic nano composites, delivery systems, and imaging. Example: to control the injection of stem cells-based therapeutics and to deliver medicinal compounds locally, to restore damaged tissue and local signalling cascades. These nano-composites are attached to the implant, at locations prescribed by clinicians, which depends on each patient's case.

5. Renal Prosthetics (1st generation): Set of sensors and micro actuators that are connected to a control system (microchip). Biomaterials and biocompatible microchips are indicated in the Description. Microelectronics (sensors) are used to capture physiological signals and to take measurements (e.g., real time dosage of creatinine in both urine and blood, real time dosage of acid uric in both blood and urine, monitoring of inflammatory biomarkers, and calculation of nephrons' filtering capacity). The AI-control system deployed in a microchip (microcontroller) forecasts renal failure using these variables and responds to imminent threats by sending signals to the actuators, to maintain local homeostasis at acceptable levels. This implant via AI-control coordinates (i) real-time renal function monitoring, (ii) drug delivery, (iii) imaging, and (iii) regenerative tissue therapeutics (e.g., based on stem cells technology) in patient with reduced renal capacity, without the indication of nephrectomy. The diseased organ is constantly monitored and treated.

6. Renal Prosthetics (2nd and 3rd generations): 3D printed reconstructed structure replicating the patient kidney's target volume, whose design is personalised, according to 3D reconstructed and segmented CT/MRI data, without following anatomic patterns, precisely. This structure contains multiple chambers, filters, and valves to filter the blood and pump both clean blood and residual fluid (artificial urine) using a target chamber volume. To adopt the system with important functional capabilities, if surrounding impaired tissue or local homeostasis need to be treated, therapeutic components (e.g., drug delivery and stem cells technology) are used. Sensors are strategically placed to monitor the prosthetics functionality for mitigating faults, measure physiological flow drivers (e.g., gradient of pressure) and concentration of blood compounds (e.g., concentration of dialysed uric acid) to control filtering and pumping mechanisms, along with concentration of chemical compounds leading to physiological impairment such as hypocalcaemia, to feedback safety and alert mechanisms.

7. Neural Implant (1st generation): Nano-composites combined with sensors and actuators that are controlled by AI technology, for signals analysis, dynamically monitoring and triggering the delivery of chemicals to brain tissue. Three variations of the model are presented. They have a similar structure, varying only in target signalling cascade (or group of neuronal cells to be treated) and needed compound to be delivered. The materials used to encapsulate implanted devices and to form in artificial shells (artificial tissues) are indicated in the Description. Microchips used in sensors for electrical signals registration probes, in imaging, and in AI-based implant's control are also indicated in the Description. These nano-composites combined with sensors, actuators and an AI-control system monitor, analyse and control/adjust chemical reactions and the relevant signalling cascades. The variations of the model are as follows. Model 1 is a bio-implant that collects local electrical and biochemical signals and use AI technology to drive immune assays for biomarker determination and knockdown of diseased signalling networks. The target is amyloid and/or tau, modulating diseased signalling cascades, to mitigate Alzheimer plaque build-up. Model 2 is a bio-implant for local physiological monitoring, dynamically delivering compounds, to mitigate excitotoxicity caused by imbalance in expression of neurotransmitters. Model 3 is a bio-implant for drug delivery and disease progression follow-up (dynamically capturing signals during treatment, which are analysed in real time, to monitor progress and trigger local drug administration accordingly, in response to different physiological responses). This model is used for tissue recovery, following for instance brain injury caused by strokes or cranial traumatism resulting from road accidents, and other critical episodes.

8. Neural Implant (2nd generation): A set of signals transmitters leveraging electromagnetic dysfunction. The transmitters communicate with an AI platform for signals processing and analysis. Again, the materials used to encapsulate implanted devices and to form artificial shells (artificial tissues), microchips used in sensors for electrical signals registration probes, in imaging, and in AI-based implant's control are indicated in the Description. The transmitters coupled with AI microcontrollers monitor, analyse and control/adjust electromagnetic signals that are related to functional impairment like visual dysfunction and hearing loss. There are two variations of the model, as follows. Model 1 is a biocompatible electrical encoder-decoder implant that supports the transmission of visual information from the retina to the brain, when the process is compromised by optical nerve damage, mitigating vision loss (e.g., in patients suffering from glaucoma). Model 2 is a biocompatible electrical encoder-decoder implant that supports the transmission of sound from the cochlea to the brain, when the process is compromised by auditory nerve fibre damage, mitigating hearing loss.

9. In all the implants here presented, a complete AI platform was developed to control the system, to design the implants in a personalised manner based on patients' CT/MRI images, and to generate updates for the algorithms deployed on the microcontrollers (i.e., the algorithms that analyses signals and control the implants). This AI platform also contains a virtual environment to plan computer-guided robotic surgery. The signals analysed are collected into variables. These variables are divided into four main groups according to their usage (indicated in the Description).

10. As an additional feature, the implants send recorded signals to (i) a light-based alert microchip implanted in the patient's wrist, (ii) a computer located in the hospital where the patient is treated, and (iii) the patient's mobile device. This is for safety and to store signals for software updates.
